(19) United States
(12) Patent Application Publication
(10) Pub. No.: US 2006/0257378 A1
(43) Pub. Date: Nov. 16, 2006

(54) Adaptive SOL-GEL immobilization agents for cell delivery

(75) Inventors: Eric T. Crumpler, Miami, FL (US); Martha C. Vallejo, Sunrise, FL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: FLORIDA INTERNATIONAL UNIVERSITY BOARD OF TRUSTEES, Miami, FL (US)

(21) Appl. No.: 11/385,384

(22) Filed: Mar. 21, 2006

Related U.S. Application Data
(60) Provisional application No. 60/663,649, filed on Mar. 21, 2005.

(51) Int. Cl. A61K 35/12; A61K 9/14

(52) U.S. Cl. 424/93.7; 424/486

(57) ABSTRACT

The present invention relates to formulations which form hydrogels at physiological temperatures. More specifically, the present invention relations to formulations having a prepolymer, an additive, and a temperature protectant, wherein the prepolymer and additive polymerize at physiological conditions to form a hydrogel. The formulations can further comprise cell suspensions such that the formation of the hydrogel leads to encapsulation of the cells in the hydrogel. The cells can then be slowly released into the surrounding environment of the hydrogel, allowing for more effective cell delivery.

### CROSS-REFERENCE TO RELATED APPLICATIONS

This application claims the benefit of U.S. Provisional Application Ser. No. 60/663,649, filed Mar. 21, 2005, which is incorporated by reference herein in its entirety.

### INTRODUCTION

Cardiovascular disease remains the leading cause of morbidity and mortality in the United States, claiming the lives of nearly 39% of the more than 2.4 million Americans who die each year. Individuals who suffer an acute myocardial infarction (MI, or heart attack), and manage to survive are at high risk for scar tissue formation. Loss of viable myocardial cells results in increased wall stress in the remaining myocardium, which ultimately leads to left ventricular dilatation and heart failure. Current medical therapies for acute myocardial infarction including cardiac transplantation, coronary artery bypass grafting and biological and mechanical cardiac assist devices have elucidated some of the potential problems but most of the limitations remain unsolved. Thus, in virtue of the limited efficacy of these current treatments, new restorative options such as cellular cardiomyoplasty (CCM) are emerging. CCM consist of the implantation of immature progenitor cells into the scar area which compensate for the cell loss and provide a new therapeutic possibility for infarct treatment. Although several cell types have been transplanted into injured myocardium, skeletal myoblast (also known as satellite cells) offer the best potential for clinical application due to their unique features. One major challenge when applying this technique is retention due to the high attrition rate (90%) of implanted cells, which is partially attributed to the inflammatory response that occurs after an injury and to the physical factors involved during cell delivery. Furthermore, in direct injection of dissociated cells, it is difficult to control shape, size and location of the grafted cells. Cellular delivery mechanisms that have already been developed are limited due to their low retention rates for the encapsulated cells, are difficult to remove after the cells are delivered. A variety of natural and synthetic polymer gels such as calcium alginate, agarose, chitosan and polyacrylate gels have been used to encapsulate various cell types for tissue regeneration purposes. However, these polymer gels are difficult to remove from the area once they have deposited their encapsulated cells.

Unlike some tissues or cells in the body, the heart cannot regenerate cells lost through apoptosis or necrosis. Therefore, individuals who suffer a large myocardial infarction (MI) most often develop congestive heart failure, due to the extreme loss of heart tissue. Unlike smooth muscle, cardiomyocytes that survive the injury of a MI are not capable of reentering the cell cycle, which inhibits their ability to proliferate. Therefore, the necrotized cardiomyocytes are replaced by connective tissue scar resulting in a progressive loss of functional myocardium and leading to a successive reduction in cardiac performance. Furthmore, the adult heart lacks a substantial supply of resident cardiac progenitor or stem cells that allows efficient cardiomyocyte regeneration after injury or infarction.

Present treatment alternatives for acute MI and successive failure include conventional revascularization either by coronary artery bypass grafting or by percutaneous coronary interventions, cardiac transplantation, and medical management; however, these treatments often provide unsatisfactory repair or maintenance. In the case of conventional revascularization patients are restricted by two major limitations: the target area requires the presence of both suitable vessels, and viable myocardium. Lack of either one renders this type of treatment impossible or ineffective. In the case of transplantation, there are severe donor shortages and problems with immunosuppression. Since these current alternatives offer limited efficacy and inadequate cardiac function repair other therapeutic options must be pursued.

Tissue engineering (TE) has been developed to provide a new solution for the restoration and improvement of tissue function that has been lost to trauma or disease. Conceptually, tissue engineering systems are based on three major components: (1) isolated cells that have the capacity to differentiate and expand in vitro, (2) biomaterial polymeric scaffolds that function as carriers to promote cell activity and provide a three dimensional (3-D) template for tissue development, and (3) bioreactor culture vessels that supply the optimal in vitro environment for the cell-scaffold matrix to develop into functional tissue.

Cells used in TE can be obtained from different sources including primary tissue and cell lines. Primary tissue can be allogeneic (from different members of the same species), xenogeneic (from different species) autologous (from the same individual) and syngeneic (from a genetically identically individual). At present, the use of xenogeneic and allogeneic cells in TE applications is limited due to the need for host immunosuppression. Thus the majority of TE experiments involving cell/polymer construct technology employ autologous cells. These cells are isolated from the patient and further culture and expanded in vitro, under specific conditions that resemble the biochemical and physical interactions essential for in vivo tissue growth and development. Once the cells have been expanded in cell culture, they are seeded on a 3-D polymer scaffold for incubation either in vitro or in vivo to encourage cell differentiation, and to provide the support necessary to generate organized tissue structures. The scaffold supplies the three-dimensional structure that enables cell attachment and tissue growth, and the reactor vessel supplies the cells-polymer matrices with an enhanced environment so they can evolve into functional tissue.

Polymer scaffolds are major components of the TE system due to their role as 3-D matrices for tissue growth and support. Scaffolds are porous materials fabricated from natural or synthetic materials. The ideal scaffold for TE should comply with several requirements: high porosity to allow uniform cell distribution, controlled degradation at the same rate of in vitro tissue regeneration, and high biocompatibility. Poly (glycolic acid) (PGA) and their copolymers represent the most widely used biodegradable and biocompatible synthetic polymers in medicine. PGA is a crystalline, hydrophilic linear aliphatic polymer that degrades through hydrolysis of ester bonds and bulk erosion. It loses much of its mechanical strength in a period of 2-4 weeks in a fluid pH 7 at 37° C. (i.e., physiological conditions). Surface modification of PGA scaffolds by hydrolysis has been studied for increasing cell seeding density and improving biomaterial-cell interactions.

Three general TE therapeutic strategies have been studied and employed thus far for cardiac applications: (1) implantation of cell suspensions needed for function directly into the heart, (2) implantation of mature 3-D tissues assembled in vitro and obtained from the combination of dissociated cells seeded onto polymeric 3-D scaffolds, and (3) cells placed or contained within a matrix crafted from synthetic or naturally occurring materials.

Isolated cell therapy denotes a form of in vivo myocardial TE. For optimal effect on cardiac function, cellular therapies must consider not only the identity of the transplanted cells but also the method of cell delivery. Cell transplantation is based on two major assumptions: (1) HF arises when a significant number of cardiomyocytes has been lost, and (2) cardiac function can be enhanced by reestablishing new cell pools into these areas of necrotic myocardium. The main requirement to improve cardiac function is that these new cell pools posses contractile properties. Several cell types have been transplanted into injured myocardium: fetal and adult cardiomyocytes, autologous skeletal myoblast, smooth muscle cells, and mesenchymal stem cells have been transplanted into injured myocardium.

Skeletal myoblasts are located under the basal membrane of skeletal muscle fibers. These type of cells offer several features for clinical applications. First, they have an autologous source overcoming problems related to availability and ethics. Second, the isolated myoblasts can proliferate well in vitro offering the advantage of wide scale up. Third, they are committed to a well-differentiated myogenic lineage extensively eliminating the possibility of tumor development. Finally, they are highly resistant to ischemia which is a key advantage due to the hypoxic environment of post-infarct areas where they are implanted. To date, myoblast transplantations have been mainly achieved by injection of myoblast cell suspensions into mature skeletal muscle. These single cells have been shown to fuse with the host myofibers.

Isolated mammalian cells and tissues have showed vast applicability in both medicine and biotechnology. The majority of mammalian cells are anchorage-dependent; therefore the optimal immobilization matrix should not only protect them from host immune responses but also provide them with the suitable environment for attachment and proliferation. Cell and tissue immobilization systems are generally classified in three categories: (1) entrapment of cells in polymer gels or porous matrices, (2) adhesion of cells on microcarrier surfaces and (3) capture of cells behind membranes.

Block copolymers as hydrogels have been shown useful in several medical applications. Cartilage formation was achieved in a nude mice animal model after subcutaneous delivery of bovine chondrocytes in a PEO-PPO-PEO based hydrogel. Cartilage formation was also achieved in a porcine animal model by encapsulating autologous chondrocytes using PEO-PPO-PEO based formulation hydrogels. PEO-PPO-PEO block copolymers have been shown useful as novel functional molecules for gene therapy. Peripheral nerve reconstruction using PEO-PPO-PEO based formulation hydrogels and poly-(glycolic acid) (PGA) scaffolds in a nude mice animal model has also been demonstrated.

The hydrogels used for cell delivery have not been able to achieve high cell retention rates (i.e., greater than 15%) nor have they been biodegradable without the use of an enzyme or other additive to aid in degradation. What is needed is a new means of delivering cells to an area of interest which has a high degree of cell retention, allows for controlled release of the encapsulated cells, and is biodegradable after performing its function.

### SUMMARY OF THE INVENTION

Disclosed herein is a technology which provides biodegradable/thermoreversible hydrogels that can encapsulate a cell of interest and be delivered to an area of a subject and initiate tissue regeneration. Encapsulation of the cells can occur when the hydrogel is formed in the presence of a cell suspension. The cells are sequestered within a semi-permeable membrane of the hydrogel and isolated from the immune system, protecting them from normal host defenses. The encapsulation matrix (i.e., the hydrogel) provides a mechanical support by immobilizing the cells and keeping them uniformly distributed throughout the targeted cell compartment, as well as allows optimum nutrient and oxygen diffusion. The thermoreversible nature of the hydrogels disclosed herein, further provide a convenient means of supplying the hydrogel to the subject. The difference in temperature between ambient temperatures and a subject's body temperature induces the formation of the hydrogel, thereby ensuring that the hydrogel releases cells into the surrounding environment in an efficient manner.

One aspect of this investigation provides formulations of an injectable, biodegradable, mixture for enhanced retention of cells in an in vitro model that provides a platform for in vivo cell retention. The materials are optimized for retention of cells, such as skeletal myoblasts, in both static and dynamic environments. The resulting hydrogels from the formulations allow for greater cell retention rates over those of previous matrices reported, e.g., of greater than 20% cell retention.

The formulation of this novel hydrogel allow for the development of newer generations of multi-component therapies for repairing damaged myocardial tissue without risk of thrombolytic migration complications. Second, this hydrogel matrix provides a suitable environment for cell encapsulation and delivery. Third, restricted physiological gelation rates coupled with controlled degradation provide high bioretention rates and increase the potential for engraftment into damaged or diseased tissue. Fourth, the hydrogel matrix is biodegradable without the need of enzyme additives, which allow for safe delivery and implantation of the matrix without concern for the ability to remove or degrade the matrix after sufficient time for cell delivery.

If the formation of the hydrogels occurs in the presence of a cell suspension, the cells will be encapsulated in the resulting hydrogel matrix. The characteristics of the hydrogel dictate the rate of release of the cells into the surrounding environment and the level of initial cell retention.

### BRIEF DESCRIPTION OF THE DRAWINGS

FIG. 1 shows the gelling temperatures of various formulations of Pluronic® F127 and temperature protectants, such as PBS, HTS, HTS/Saline, and control (water);

FIG. 2 shows the gelling point of various formulations of Pluronic® F108 and temperature protectants, such as PBS, HTS, and HTS/Saline;

FIG. 3 shows the transition time of a formulation comprising Pluronic® F127 to form a hydrogel at various temperature;

FIG. 4 shows the spreading distance of a formulation prior to hydrogel formation at various temperatures;

FIG. 5 shows the cell viability within a hydrogel at various times and temperatures;

FIG. 6 shows pH measurements of hydrogels of different temperature protectants; and

FIG. 7 shows cell retention levels with hydrogel versus no hydrogel in an in vivo model.

### DESCRIPTION OF THE PREFERRED EMBODIMENTS

Disclosed herein are formulations that can be used for the delivery of cells to a specific area of a subject. These formulations comprise a prepolymer, a temperature protectant, and an additive. The resulting formulation is polymerized via cross-linking of the additive with the prepolymer to form hydrogel matrices. The role of the temperature protectant is to provide stability of the formulation prior to formation of the hydrogel. Because the formulations disclosed herein are thermally polymerized, the temperature of the formulation prior to introduction to a subject should be less than that subject's physiological temperature, typically stored at low temperature, i.e., temperatures less than room temperature, typically about 0 to about 20° C.

The formulation can be injected into a subject using a solution which further comprises cells, such as skeletal myoblast cells, and the temperature of the body of the subject will induce a polymerization that creates a matrix for the slow release of the cells to the surrounding area of the subject. Alternatively, the formulation can be implanted into a subject using surgical techniques and the hydrogel results in a similar manner (i.e., via thermal cross-polymerization due to the temperature of the subject). In another embodiment, the delivery of the formulation and cells can occur through a catheter or through other non-invasive means.

Hydrogels are three dimensional (3D) hydrophilic polymeric networks capable of imbibing and retaining large amounts of water or biological fluid. In order to maintain their 3D structures, polymer chains of hydrogels are usually chemically or physically crosslinked. The amounts of liquid that hydrogels are capable of withholding ranges from 20% to 40% of their total dry weight. One of the inherent properties of these biomaterials is their ability to swell in liquid solutions and shrink in their absence. They are elastic solids because their ability to return to their original configuration after being deformed. The main areas of hydrogel applicability as biomaterials include synthetic wound care coverings, dental materials, implants, injectable polymeric systems, ophthalmic applications, and encapsulation delivery. Hydrogels can be classified as naturally derived, such as collagen, alginate, agarose, and chitosan or synthetic, such as poly (ethylene glycol), poly (vinyl alcohol) (PVA) and triblock copolymers of polyethylene oxide (PEO) and polypropylene oxide (PPO), such as PEO-PPO-PEO. Although the majority of encapsulation matrices have been naturally derived materials, synthetics present several features such as high purity, high availability, and they can be easily tailored to fit different cell types.

Chemical gels possess polymer chains that are connected through covalent bonds, whereas physical gels have polymer chains linked by non covalent bonds or cohesion forces, such as Van der Waals interactions, ionic interaction, hydrogen bonding, or hydrophobic interaction. Due to the nature of CCM procedures, the matrix selected along with the cells must be delivered into the heart in a minimally invasive way, either by direct injection or through a catheter. Furthermore, the matrix should withstand the pressure and movement in the heart without rupturing or migrating to other parts of the body. Physical hydrogel-based systems satisfy this criterion largely by remaining malleable and complying to contortions of the surrounding environment. Since the bonding between polymer chains is reversible, physical gels possess sol-gel reversibility. Some additional advantageous properties offered by these biomaterials include their swelling behavior, which provides an aqueous environment that resembles natural tissue and allows for good transport of nutrients to cells crucial for cell viability, as well as transport of waste out of the hydrogel. Furthermore, the soft, rubbery nature of hydrogels causes minimal mechanical and frictional irritation to the surrounding tissue. Thus, there is minimal pain involved and little damage caused to the mucous membranes or to the intima of the blood vessels resulting in the prevention of infections or thrombus formation. Moreover, hydrogels present low interfacial tension with surrounding biological fluid and tissues reducing the driving force for protein adsorption and cell adhesion.

Physical hydrogels can further be grouped in two categories: (1) conventional hydrogels-light crosslinked hydrophilic polymers which swell in the presence of water but do not dissolve or react to environmental factors and (2) stimuli responsive hydrogels-polymeric gels capable of reversible contraction and expansion under physico-chemical stimuli. These types of polymer gels respond to small changes in environmental conditions such as pH, temperature, electric field, ionic strength, salt type, solvent, external stress, light, and combinations of these. Hydrogels that are induced by temperatures changes are called thermoreversible gels.

Triblock (ABA) copolymers of poly(ethylene oxide) and poly(propylene oxide), often denoted PEO-PPO-PEO are commercially available nonionic surfactants, such as from BASF (called Pluronic®) or from ICI (called Poloxamers). These triblock copolymers are produced by the sequential addition of propylene oxide (PO) and ethylene oxide (EO) to a water-soluble low molecular weight propylene glycol, a poly (propylene oxide). The molecular weight of each block of the triblock copolymer can be controlled to provide the desired end properties of the prepolymer. A higher ratio of PEO to PPO will result in a more hydrophilic prepolymer; a higher ratio of PPO versus PEO will result in a more hydrophobic prepolymer. The charactertistics of the triblock copolymer also influence the polymerization conditions for the formation of the hydrogel.

The prepolymer of the present formulation is a low molecular weight polymer of water soluble components, such as the triblock copolymer of poly(ethyleneoxide)-poly(propyleneoxide)-poly(ethyleneoxide) (PEO-PPO-PEO). Other suitable polymers include, but are not limited to, polyethylene glycol, polyvinyl alcohol, polyvinyl acetate, and polyacrylamide, and collagen. The molecular weight of the prepolymer ranges from about 9200 to about 15,000 Da. The prepolymer needs to have a free reactive group in order to react with the additive(s) to form the hydrogel matrix of the present invention. The amount of prepolymer in the formulation can range from about 5 to about 25% of the total weight of the formulation. Preferred ranges include about 10 to about 18 wt %, and a particularly preferred range is about 16wt %. For a prepolymer of PEO-PPO-PEO for use in the disclosed formulations, a ratio of PEO to PPO about 1.65:1 to about 3:1 is preferred; more preferred is a ratio of about 2:1.

PEO-PPO-PEO block copolymer solutions exhibit temperature-dependent micellization and are capable of forming gels. At low concentrations and/or solution temperatures, these triblock copolymers exist as individual coils also called unimers. Upon temperature and/or solution concentration increases, the copolymer molecules aggregate to form thermodynamically stable micelles. This micellization occurs due to the dehydration of the hydrophobic PPO block with temperature. The micelles formed are spherical, and they are composed of a dehydrated inner core of PPO with an outer shell of hydrated, swollen PEO chains. These micelles of PEO-PPO-PEO are suitable for crosslinking and can then be manipulated to contain cells for a cell delivery system.

Pluronic® F127 has a relatively long chain length (Mw: 12,600) and an appropriate n/m ratio (about 1.5/1, that is 100 EO units-65 PO units-100 EO units), which results in a roughly 70 wt % ethylene oxide (EO) content. This design of F127 ensures both good water solubility through the high content of EO and a high capacity for hydrophobic association through the relatively long PPO block. Pluronic® F108 (where the molecular structure is 133 EO-50 PO-133 EO) is another Pluronic polymer with a highest molecular weight of 14.6 kDa. The EO content and n/m ratio of F108 are 80 wt % and 2.8, respectively. Thus, the micellization, micellar structure, and phase behavior of F108 differ from those of F127. For example, F108 would have higher water solubility and smaller micellar core sizes than F127.

The temperature and concentration at which micelles start forming are described as the critical micelle temperature (cmt), and the critical micelle concentration (cmc). Both cmc and cmt decrease with an increase in the copolymer PPO content or molecular weight. The cmt is inversely proportional to the concentration whereas the concentration increases; the cmt decreases. The reverse thermo responsive behavior of these types of polymers is due to their liquid crystalline organization based on self-assembly.

Moderate high-concentration copolymer solutions exhibit low viscosity at ambient temperature, and increase their viscosity with rising temperatures, producing a semi-solid gel at body temperature. The prepolymer can be tailored to exhibit specific characteristics for various applications by adjusting the molecular makeup of its components. Gel transition temperatures, micelle characteristics, and the like all can be programmed by choosing, for the case of PEO-PPO-PEO triblock copolymers, the lengths and ratios of each block of the prepolymer. In some embodiments, suitable prepolymers are those which are able to form micelles at physiological temperatures.

The temperature protectant of the present invention is a solution that protects the stability of the formulation at low temperatures, such as about 0 to about 20° C., used to preserve the formulation prior to injection or placement into a potential subject. The temperature protectant can be those provided by commercial sources, such as BioLife Solutions, Inc. (Owego, N.Y. USA). Typical temperature protectants include BioLife's HypoThermosol® and CryoStor® series of media. Other media that play the same role as those of BioLife may be used in the formulations of the present invention. The amount of temperature protectant present in the formulations of the present invention is about 4 wt % to about 8 wt %. Preferred amounts are about 4 to about 6 wt %.

The additive of the present invention is a component that is capable of cross-linking the prepolymer into a hydrogel matrix. The choice of additive for the formulation will be dependent upon the identity of the prepolymer components. A preferred additive is polyethylene glycol (PEG). The PEG is able to noncovalently interact with the prepolymer component to form a cross-linked hydrogel. A preferred combination of prepolymer and additive is a triblock copolymer of PEO-PPO-PEO and PEG. The molecular weight of PEG in the formulation is about 2000 to about 8000 Da, and preferably about 3000 to about 5000 Da, and more preferably about 5000 Da. Other preferred additives include polyglycolic acid and polylactic acid. The amount of additive in the formulations of the present invention range from about 0.2 wt % to about 1 wt %, and preferably are about 0.5 wt % to about 0.75 wt %. Too much additive will result in a hydrogel that is too cross-linked and will not allow slow dispersion of the cells in a controlled fashion, and too little of the additive will result in a hydrogel that does not hold the cells or allow for the desired slow, controlled delivery of cells suspended in the hydrogel matrix.

The formulations of the present invention can be mixed with a suspension of cells for controlled delivery or controlled release within a subject, such as a human or animal subject. The cells can be any which may be of therapeutic use to the subject. The selection of cell type is based on the desired application for the final hydrogel, and may, for example, comprise smooth muscle cells, endothelial cells, epithelial cells, fibroblasts, myoblasts, bile duct cells, pancreatic islet cells, thyroid cells, parathyroid cells, adrenal cells, hypothalamic cells, pituitary cells, ovarian cells, testicular cells, salivary cells, chondrocytes, hepatocytes, enterocytes, nerve cells, cardiac cells or kidney cells. In one embodiment, the cells comprise myoblasts for use in the repair of damaged muscle tissue. The concentration of the cell suspension can be about 0.5 million to about 10 million cells/mL. The amount of cells encapsulated within a hydrogel will be dependent upon the concentration of the cell suspension. Typically, the amount of cells encapsulated will be about 0.75 million to about 1.3 million cells/hydrogel.

The cells are incorporated into the hydrogel upon crosslinking of the prepolymer with the additive. The cells, in pellet form, are first resuspended into a solution comprising the prepolymer, temperature protectant, and additive. Delivery of the mixture to a subject induces crosslinking of the prepolymer with the additive and encapsulates the cell suspension within the resulting hydrogel. Providing a high seeding density of viable cells allows a greater number of cells to expand and release extracellular matrix molecules into the surrounding tissue of the hydrogel. The formulations of the present invention can provide cell retention levels from about 20% to about 40% with respect to number of starting cells versus number of remaining cells in the same area over a period of time.

Another aspect of the present invention provides a method of delivering the formulations to a subject in need. The formulations can be delivered via a catheter or through surgical implantation. The physiological conditions of the subject induce the thermal polymerization of the prepolymer and the additive to form the hydrogel. When the polymerization is done in the presence of a cell suspension, the cells are encapsulated in the hydrogel and can be released slowly into the subject to allow for an increased efficacy of the therapy requiring the cell delivery. In one embodiment, the cells encapsulated by a hydrogel of the formulations of the present invention are skeletal myoblasts.

Still another aspect of the present invention is a method of regenerating tissue via the delivery of cells through a controlled manner using a hydrogel of the present invention. Encapsulated cells in a hydrogel are slowly released into the surrounding environment of a subject which facilitates tissue regeneration.

Additional aspects and details of the disclosure will be apparent from the following examples, which are intended to be illustrative rather than limiting.

### EXAMPLES

Materials and Methods

c2c12 cells were obtained (American Type Culture Collection, Rockville, Md.) in an ampoule containing 1×10⁶. This is a sub-clone from myoblast line established from normal adult C3H mouse leg muscle. The c2c12 cell line differentiates rapidly, forming contractile myotubes and producing characteristic muscle proteins.

Dulbecco's modified Eagle (Gibco, Grand Island, N.Y.) was supplemented with 10% characterized fetal bovine serum (FBS) (Hyclone, Logan, Utah) and 1% penicillin-streptomycin-glutamine (Gibco, Grand Island, N.Y.).

A rotary cell culture system (RCCS) was obtained from Synthecon Inc., Houston, Tex. The RCCS is a horizontally rotated, bubble free culture vessel with membrane diffusion gas exchange. The system consists of a 4-station rotator base which incorporates 4 independent variable speed motor drives capable of holding 4 single 55 mL disposable vessels.

Polyglycolic acid unaligned scaffolds (Synthecon Inc., Houston Tex.) were obtained in 10 cm×10 cm sheets. Individual pieces were cut from the sheet to the desired shape and sizes.

Pluronic® F127 and F108 (Prill) were donated by BASF, Mount Olive, N.J. Hypothermosol® was obtained from Hyclone, Logan, Utah, PBS was supplied by Gibco, Grand Island, N.Y., and 0.9% Saline (Baxter Healthcare Corporation, Deerfield, Ill.) was kindly donated by Dr. Juan Franquiz.

Hydrogel Formulation

Two triblock copolymers, Pluronic® F127 and F108 were used as stock materials for the preparation of the hydrogel matrices. An appropriate mass to volume (m/v) ratio was formulated for desired experimental conditions. The hydrogels were prepared using the cold method in which the polymer/solvent mixture was cooled to 2-5° C. until a homogenous solution was formed usually between 24-48 hours (Schmolka, 1972). The solubility of the hydrogels was tested in various solutions including, 0.9% saline, phosphate buffered saline (PBS), and Hypothermosol®.

Optimal composition of a 20 mL formulation for hydrogel formation for delivery to a subject via surgical means (16% by weight) were as follows: 3752 mg Pluronic® F127, 1.5 mL Hypothermosol®) (HTS), 17.5 mL MyoChoice™ with Myobooster™ Media (Bioheart Inc.), 1 mL cell suspension added after neutralized trypsinization.

Optimal composition of a 20 mL formulation for hydrogel formation for delivery to a subject via catheter or other non-invasive means (16% by weight) were as follows: 3752 mg Pluronic® F127, 2.0 mL Hypothermosol® (HTS), 17.0 mL MyoChoice™ with Myobooster™ Media (a specialized media available from BioHeart, Inc.), 1 mL cell suspension added after neutralized trypsinization.

### Composition of 12-24 wt % 20 mL Formulations Tested for Both Surgical and Non-Invasive Delivery

| % weight of | Amount of | Volume of | Total Media |
|---|---|---|---|
| prepolymer | Pluronic F127 (mg) | HTS (mL) | Volume (mL) |
| 12% | 2686.00 | 1 | 19 |
| 14% | 3219.25 | 1 | 19 |
| 16% | 3752.00 | 1 | 19 |
| 18% | 4335.36 | 1 | 19 |
| 20% | 4937.50 | 1 | 19 |
| 24% | 6235.00 | 1 | 19 |

200 μL of different concentrations of F127 can be used and the volume of the additive polyethylene glycol (PEG) (molecular weight about 5000 Da) is adjusted with specific formulations, to allow for different gelation when the application of the formulation is delivered surgically versus a non-invasive means, e.g., via catheter.

Hydrogel Characterization

Several experiments were conducted in order to identify the optimal injectable matrix for cardiac tissue regeneration. Gelling point, gelling times, spreading distances, pH measurements, cell viability in the hydrogel matrix, DSC, and SEM were evaluated in order to characterize the hydrogel matrix

The tube inversion method (Jeon et al., 2002) was used to determine the gelling point (Gₚ) for both F108 and F127 Pluronic® triblock copolymers. Three different solvents were used to prepare the hydrogels: 1) Hypothermosol®, 2) PBS (phosphate buffered saline) and 3) a Hypothermosol®-saline mixture. Hydrogel concentrations ranged from 9-23 wt %. Measurements were assessed at 2° C. increments from temperatures ranging from 5° C. to 47° C. A 5 ml hydrogel sample (cooled at about 2-5° C.) was released in a glass vial sealed with Teflon tape to avoid leakages. The vial was placed in a digital water bath for at least 10 minutes before each measurement to ensure temperature equilibration. Variation from a mobile state to an immobile state was determined by inverting the vial. Gels were defined as those samples in which the solution did not flow to the bottom of the vial upon inversion within 5 minutes, whereas sols were defined as those that promptly flowed to the bottom of the vial within 5 minutes after inversion. According to the results obtained from this experiment, the triblock copolymer had sufficient properties and follow up studies were developed to create an optimal hydrogel matrix with this copolymer.

In order to asses the time needed for the hydrogel to go from a mobile state to an immobile one (Gₜᵢₘₑ, i.e., gelling time), a 5 mL hydrogel sample (cooled at about 2-7° C.) was taken into a glass vial. The glass vial was covered with parafilm tape and placed in a digital water bath. Measurements were evaluated at 1° C. increments from 35-40° C. Temperatures were allowed to equilibrate for 10 minutes before taking any measurements. Triplicate readings were obtained for each of the temperatures, and the average reading was considered to be the Gₜᵢₘₑ. All samples were randomized.

The spreading distance (Sₚ) measurement was included in order to asses the area coverage of the hydrogel before turning into a gel. Glass vials were placed in a digital water bath for 10 minutes to allow for temperature equilibration. When the desired temperature was achieved, a 0.5 mL hydrogel sample (cooled at about 2-7° C.) was released with a 1000 μL pipette in to the glass vial (about 45° angle). Measurements were evaluated at 1° C. increments from 35-40° C. The Sₚ was measured to be the distance traveled by the hydrogel before stop flowing along the glass vial. Triplicate readings were obtained for each temperature, and the average reading was determined as the Sₚ. All samples were randomized.

Cell viability in the hydrogel matrix was assessed by trypan blue exclusion (1:1 dilution). Triplicate readings were obtained at times zero, thirty minutes, and one hour. Viability was tested with the hydrogel at room temperature and on ice.

A digital pH meter was used to obtain the hydrogel pH at three different concentrations: 9, 17 and 23 wt %. Two different solvents were tested: PBS and Hypothermosol®-saline.

Differential Scanning Calorimetry (DSC) was used to measure the thermal transition of the hydrogel as a function of temperature. Four samples were obtained from concentrations 14, 17, 19, and 22 wt %. Sample volumes of 10 μL were scanned in hermetically sealed aluminum pans at a heating and cooling rate of 3° C./min (from 0 to 40° C.) with an empty pan as reference.

A scanning electron microscope (SEM) (JEOL, JSM-5910 LV) was used to analyze the physical morphology of the hydrogel as well as its spreading capacity and its degradation properties. The prepared specimens were gold coated before mounted in the SEM. For this purpose two 200 μL hydrogel aliquots were loaded into two PGA scaffolds via syringe injection. The first scaffold was immediately fixed with HMDS after loading the hydrogel (t=0 min). The remaining scaffold was placed into a bioreactor vessel containing 50 mL supplemented growth medium. The bioreactor was rotated at 25 RPM for 30 minutes (t=30 min) at 37° C. and 5% CO₂. Once the time frame was completed the PGA scaffold was removed from the bioreactor and fixed with HMDS.

Cell Culture

c2c12 cell line skeletal muscle myoblast cell line of murine origin was used as an in vitro model for cell retention and proliferation studies. Cell culture was completed on 75 cm² tissue culture flasks in a humidified atmosphere of 95% and 5% CO₂. Cell count and cell viability were performed prior to any experiment. Phase contrast microscopy was used to assess cell morphology, growth and differentiation.

Frozen c2c12 cells were obtained in 1 ml vials. Cells were thawed by placing vials into water bath at 37 C, and rapidly agitating them until dilution occurred. Each vial was divided into 3-75 cm2 culture flasks containing 10 ml of warmed growth medium. At 70-80% confluency cells were subcultured, centrifuged (200×g for 5 minutes) and resuspended in 5% Dimethylsulfoxide (DMSO) (Sigma Aldrich, Milwaukee, Wis. USA)—95% growth medium. Cell concentration was adjusted to be 1×10⁶ cells/mL, and aliquots of 1 mL were transferred to 1.5 mL cryo-vials. Vials were kept overnight at −80° C. and later stored in liquid nitrogen.

First, the vials were removed and the culture medium discarded. Next, the cell monolayer was rinsed with 5 to 10 mL of Dulbecco's Phosphate Buffered Saline (PBS) to remove all traces of fetal bovine serum (FBS), which contained trypsin inhibitor. Immediately after, 6 ml of 0.25% trypsin-0.53mM EDTA (w/v) dissociating agent was added to the flask, and the resulting mixture was incubated at 37° C. and 5% CO2 for 2-5 minutes (trypsin-EDTA was be pre-warmed at 37° C. before adding to the cell layer). Cells were observed under inverted phase contrast microscope, and flasks were gently tapped to allow for cell detachment. Once the cells looked rounded and had unattached from the flasks, added 6 ml of serum supplemented growth medium to neutralize the trypsin solution. Removed the cell suspension and centrifuged for 5 minutes at 200×g to obtain a cell pellet. Next, collected the suspended cells and aspirated the supernatant. Finally, cells were resuspended into the proper amount of pre-warmed (37° C.) DMEM growth medium and further counted with the help of a hemocytometer. Cultures were prevented from becoming confluent as this depleted the myoblastic population in the culture. Cells proliferated and became 80% confluent in approximately 2-3 days. The sub-cultivation ratio was established as 1:4. Viability was also checked by using trypan blue solution.

Full media renewal was done every 2-3 days. Old medium was removed and 10 mL of pre-warmed growth medium were dispensed into each 75 cm² culture flask.

Cells in suspension were counted using a hemocytometer. A 20 μL sample cell suspension was wicked into each side of the chamber and the number of cells per unit area was counted manually.

Cell viability was determined with trypan blue exclusion by mixing equal volumes of cell suspension and 0.4% Trypan Blue stain (Gibco, Grand Island, N.Y.) and incubating for 5 min at room temperature. The viable cells were considered as those in which the plasma membrane was intact and thus not stained. Non-viable cells were considered as those with leaky plasma membranes were dye was not excluded and cells were colored blue. Cell viability was expressed as a percentage of the total cell number.

### c212 Cells and SRB Experimental Design

SRB assay results were expressed as cell numbers using a standard curve, constructed from a well plate seeded with known cell concentrations. Cells were harvested, prepared and incubated following the appropriate protocols. The seeding platform was a 48-well microtiter plate and the assay was conducted for a period of 24 hours. Cell plating densities ranged from 103 to 6600 cells per well and optical density (O.D.) was determined with a TECAN plate reader (520 nm).

Cell growth was considered as the change with time in the amount of cellular material within culture. After harvesting cells from 75 cm², they were seeded into 96-well microtiter plates for intervals of zero (considered at 24 hours), 6, 12, 24, 36 and 48 hours. Each of the 96-well plates was divided in two groups containing 600 and 800 cells per well. Absorbance readings were obtained with a TECAN plate reader.

### Cytotoxicity and Degradation Studies

c2c12 cells along with the hydrogel were immobilized in pre-heated (37° C.) 96-well plates for cytotoxicity and degradation studies. Metabolic activity, proliferation, and cell density were assessed using sulforhodamine B (SRB) assay. Cell density in the matrix (residential versus adherent cells) was analyzed for residential/adherence (R/Ad) validation of proliferation along with degradation studies. The optical density (O.D.) at 520 nm was determined using a TECAN plate reader.

Cells were harvested, prepared and incubated following protocols. Cell densities were selected to result in ˜1000 cells per well. Cells along with the hydrogel matrix and controls were incubated at 37° C. and 5% CO₂ for 24 hours. Total volumes for each well ranged between 150-250 μL distributed as follows: 1) 250 μL of growth medium, HTS/saline and hydrogel each (columns 2, 3 and 4), 2) 100 μL of c2c12 cell suspension and 100 μL of growth medium 3) 100 μL of c2c12 cell suspension, 100 μL of growth medium and 50 μL of hydrogel, and 4) 100 μL of growth medium and 50 μL of cell-hydrogel suspension. Two 96-well plates were prepared and analyzed to assess replicability. The experimental layout used is shown in Table I.

TABLE I

| | | 3 | | | | | | | | | |
|---|---|---|---|---|---|---|---|---|---|---|---|
| | 2 | 5% HTS | | 5 | | 6 | | 7 | | 8 | |
| 1 | Growth | 95% sal. | 4 | c2c12 | | Hydrogel top/ | | Hydrogel bottom/ | | Hydrogel/cell | |
| Blank | medium | Mixture | Hydrogel | cells | | cells bottom | | cells top | | mixture | |
| 1 | 1 | 1 | 1 | 1 | 9 | 1 | 9 | 1 | 9 | 1 | 9 |
| 2 | 2 | 2 | 2 | 2 | 10 | 2 | 10 | 2 | 10 | 2 | 10 |
| 3 | 3 | 3 | 3 | 3 | 11 | 3 | 11 | 3 | 11 | 3 | 11 |
| 4 | 4 | 4 | 4 | 4 | 12 | 4 | 12 | 4 | 12 | 4 | 12 |
| 5 | 5 | 5 | 5 | 5 | 13 | 5 | 13 | 5 | 13 | 5 | 13 |
| 6 | 6 | 6 | 6 | 6 | 14 | 6 | 14 | 6 | 14 | 6 | 14 |
| 7 | 7 | 7 | 7 | 7 | 15 | 7 | 15 | 7 | 15 | 7 | 15 |
| 8 | 8 | 8 | 8 | 8 | 16 | 8 | 16 | 8 | 16 | 8 | 16 |

| | |
|---|---|
| Controls | Experiment |

In order to investigate the effect of the hydrogel on the cells, three different layouts were assessed for the experiment (hydrogel-cells). First, cells were culture on the bottom of the well and the hydrogel matrix was seeded on top, next the hydrogel matrix was seeded on the bottom of the well and cells were seeded on top of the matrix, and finally a hydrogel-cell mixture was prepared and release into the wells. Before mixing, cells were centrifuged and the growth medium removed. The cell pellet obtained was suspended in the hydrogel matrix (mixture). Columns 1-3 were used as controls, and column 4 was used as the correction factor. The absorbance measurements obtained from this column were subtracted from columns 4-8 in order to normalize the results.

PGA biodegradable scaffolds (pre-warmed at about 37° C.) were used as a pseudo-in vivo model for retention studies. The cell-hydrogel injected scaffolds were grown in both statically and dynamically environments. The cultivation of R/Ad scaffolds was used as a static and dynamic in vitro test of cell viability, R/Ad assessment, proliferation, and functionality.

PGA polymer scaffolds for static and dynamic testing were cut into discs of approximately 5 mm diameter and 2 mm in thickness and modified by hydrolysis. Consequently, the scaffolds were placed in 12 well plates containing 2 mL of growth medium to promote adsorption of serum proteins, thereby increasing cell attachment. Scaffolds were kept in the incubator at 37° C. and 5% CO₂ for 5 hours prior cell seeding to allow for protein and nutrient absorption.

Skeletal myoblast were grown to subconfluence (70-80%) in T-75 cm² tissue culture flasks. The cell layer was rinsed with PBS and cells were enzymatically removed from flasks with 0.25% trypsin-ethylenediaminetetraacetic acid (EDTA) solution. Cells were then centrifuged (200×g, 5 min), counted and resuspended in a 50% Dulbecco's Modified Eagle's Medium (DMEM)-50% Hypothermosol® mixture to a concentration of 5-7×10⁶ cells/mL. Cell viability was determined by trypan blue exclusion. From this cell suspension 8 aliquots of 200 μL were obtained and placed into individual 1 ml centrifuge tubes.

Two groups were defined for this study, control and experimental. Each group was composed of 4 PGA scaffolds, from which three scaffolds were used for retention studies and one scaffold was used for SEM analysis. For the control group, 200 μL of cell suspension was injected into each PGA scaffold. For the experimental group, a cell pellet obtained from centrifugation was mixed with 200 μL of the hydrogel (cooled at ˜5° C.) until a homogenous suspension was achieved. Syringes, needles and vials used for seeding and mixing of the hydrogel were stored in the freezer prior to each experiment to avoid any gelling due temperature fluctuations.

Seeding density for both control and experimental groups was established to be approximately 1×10⁶ cells per scaffold. Using a 20 gauge needle and a 1 mL syringe, the hydrogel-cell suspension was seeded by inserting the needle tip into the scaffold and dispensing aliquots into pre-determined areas of the scaffold (total volume=200 μL). The injections were done in a designated pattern to allow for homogeneous cell distribution and coverage of the scaffold. The order in which the scaffolds were loaded was completely randomized. Finally, cells were allowed to adhere to the scaffold for 1 hour. Next, the cell/polymer constructs were placed into 25 cm² flasks for the static retention or into 50 mL bioreactor vessels for the dynamic retention.

### Bioreactors and 25 cm² Cell Culture Flasks

The cell-hydrogel PGA loaded constructs were tested in two environments: 1) static (25 cm² flasks) and 2) dynamic (bioreactors). For the static environment, 8 samples were individually placed in 25 cm² culture flasks and covered with 10 mL of supplemented growth medium. The growth medium was completely replaced every 2 days. For the dynamic environment, PGA constructs were placed in sets of two (2 controls or 2 experimental) in each bioreactor. A total of 4 bioreactors were loaded into the rotating station, and the rotating velocity was set up at 25 revolutions per minute (RPM). Bioreactor distribution was randomized in order to provide accurate statistical results. Prior to loading the PGA constructs, the bioreactors were washed with PBS for 30 minutes to remove any impurities. Medium replenishment was performed every 2 days.

### Cell Retention Quantification

Cell-hydrogel constructs were cultured for 3, 5 and 7 days in both static and dynamic environments. At the end of the designated time period, two samples (1 control and 1 experimental) were fixed in HMDS for SEM analysis. The remaining 6 scaffolds (3 controls and 3 experimental) were removed from the incubator and placed into 12 well plates containing 1.5 mL of 0.25% trypsin-EDTA solution. The plates were placed in a shaker (37° C.) for 30 minutes to remove attached cells from the PGA. Prior to conducting this experiment viability of the cells in trypsin (40 minutes) was determined. Complete removal of the cells from the construct was confirmed by light microscopic examination of the scaffold. Once the cells had unattached from the scaffolds, growth medium was added to neutralize the trypsin. The cell suspensions were individually transfer into 15 mL tubes and centrifuged at 200×g for 5 minutes. The supernatant was removed and the cell pellet was resuspended in 1 ml of fresh growth medium. A 10 μL aliquot was taken from each sample for cell viability and cell quantification assessment.

Several parameters were assessed for each sample. The total retention yield is the ratio of the number of cells harvested from the construct and the number of cells initially seeded onto the scaffold. The change in cell viability is the difference between the viability of the cells in the initial cell population (i.e. prior to scaffold seeding) and that of the cells harvested from constructs.

SEM was used to compare and analyze cellular distribution and retention between the controls and the experimental samples. After identification and characterization of a suitable hydrogel formulation, in vivo retention studies were performed by implanting a suspension of microspheres and hydrogel into healthy rabbit hearts to examine cell retention. The microspheres served as an in vivo retention model to mimic mammalian cells.

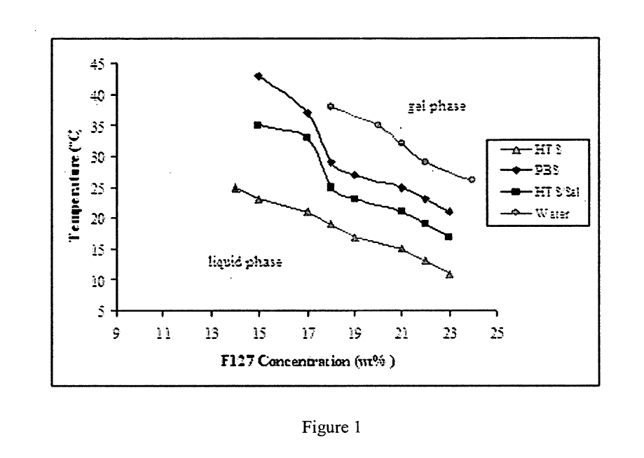

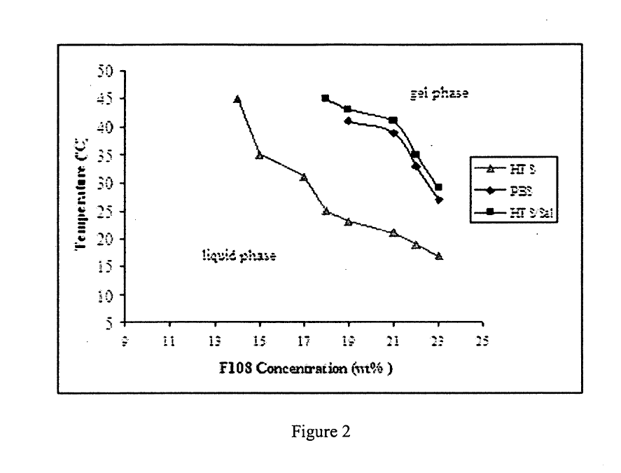

Gelling points were assessed for both Pluronics® F127 and F108 in three different solvents (FIG. 1 and 2). F127 and F108 were preferred due to their gelling capabilities. Saline and PBS were selected due to their extensive history as solvents for a variety of applications in medicine. Hypothermosol® (HTS), however, had not been used as a solvent before but as a preservation solution designed to alleviate cellular stress (ionic, osmotic, biochemical, etc.) under conditions of extended hypothermia. In view of the properties of HTS as an intracellular-like preservation solution, it was decided to test it as an optimized solvent for the hydrogel formulation.

For F127, it was observed that for concentrations below 14wt % in the temperature range studied (5-45° C.) no gel formation occurred in any of the solvents. Furthermore, for temperatures below 11° C., all the hydrogels remained in a sol state. Upon heating, samples at and above 14 wt % presented the expected transition to a gel phase at higher temperatures. This is explained by the fact that as temperature increases the PO and EO chains of the polymer progressively dehydrate and become insoluble consequentially forming micelles. Additionally, the Gₚₒᵢₙₜ trends for all the solvents reflected a temperature and concentration dependent behavior. Gelling temperature decreased with increasing concentration of F127.

All the solvents tested gave lower gel regimes than water. Solutions of F127 used in prior studies for TE and cell encapsulation techniques have been formulated in the range of 20-23 wt %, to obtain formulations that will gel at 30-35° C. (Matthew et al., 2002). Lower concentrations were needed to achieve gelling at physiological temperatures for both PBS and HTS/saline. Water was referenced in the sol-gel diagram in order to provide a valid comparison for Gₚₒᵢₙₜ assessment using the tube inversion method. Water is the most widely used solvent for hydrogels formulations specially when dealing with PEO-PPO-PEO block copolymers (Bohorquez, et al., 1999; Kabanov, et al., 2002). Gelling close physiological temperatures (35-39° C.) was attained at concentrations of 20-22 wt %. Values above these concentrations resulted in gelling temperatures close or below room temperature.

For HTS, there was no gel formation at physiological temperatures. All the concentrations formed gels at temperatures below or equal to room temperature. HTS is highly enriched with Ca⁺², Na⁺, K⁺ and Mg²⁺ among other electrolytes and buffers. The presence of salts or their anions and cations affect the cmc, shifting the whole gel region to lower temperatures. Gelling for water, PBS and HTS/saline occurred at concentrations ranging from 14-23 wt %. Similar curves were obtained for both PBS and HTS/saline solvents. The sol-gel boundary for PBS is at higher temperatures than HTS/saline. Gelling near physiological temperatures for PBS occurred between 17-19 wt %, whereas for HTS/Sal occurred at 15-17 wt %.

Pluronics® F108 presented a similar pattern to F127 in terms of general gelling behavior. Again the minimum concentration for gelling was 14wt %, all the concentrations below this point formed sols. HTS revealed gelling near physiological temperatures in the 15-17 wt % concentration range. Below this concentration gels formed close or below room temperature. PBS and HTS/Sal showed similar behavior, although this time PBS exhibited the sol-gel boundary at lower temperatures than HTS/Sal. Higher concentrations (22 wt %) were required for both solvents to form gels near physiological temperatures. At temperatures and concentrations below 14° C. it was observed that only a sol phase was present.

When comparing, both F108 and F127, F127 was chosen for further studies for hydrogel formulations. After analyzing all the solvents used for F127, it was decided that the optimal formulation was achieved with HTS/saline, due to the temperature range offer for gelling. The optimal concentration for this particular solvent was selected as 17 wt %, because it provided gelling at physiological temperatures and did not formed gels at room temperature. Lower concentrations offered a very narrow margin to achieve gelling at physiological temperatures and higher concentrations formed gels at room temperature.

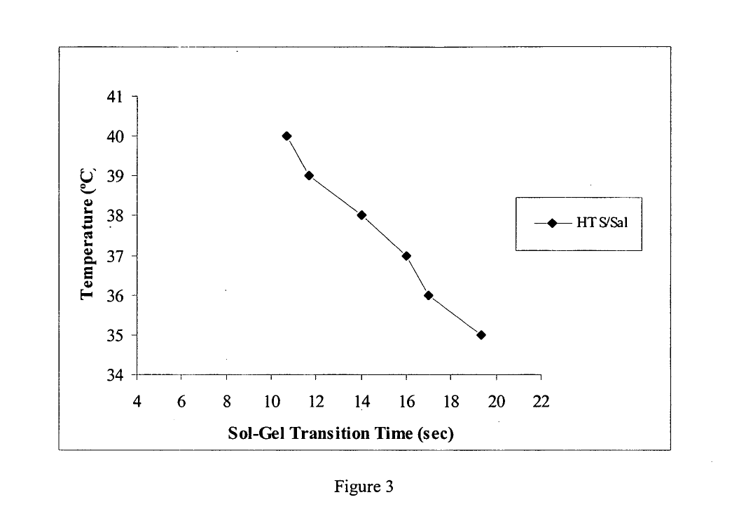

After selecting F127 in HTS/saline as the hydrogel solvent of choice for the study, further characterization was done in order to provide a better understanding of its behavior. FIG. 3 provides information on the phase transition time versus temperature. The temperature range was adjusted to 35-40° C., to cover a reasonable margin close to physiological temperatures.

From FIG. 3, it can be inferred that the mean phase transition time is inversely proportional to temperature, whereas time increases temperature decreases. From Table II, it was observed that there was an increase for the mean transition time from 11 sec at 40° C. to 19 sec at 35° C. Further comparisons to other formulations could not be assessed since this method has not been employed before.

TABLE II
Dependent Variable: Gelling Time

| | | | 95% Confidence Interval | |
|---|---|---|---|---|
| Temperature | Mean | Std. Error | Lower Bound | Upper Bound |
| 35 | 19.333 | .745 | 17.709 | 20.957 |
| 36 | 17.000 | .745 | 15.376 | 18.624 |
| 37 | 16.000 | .745 | 14.376 | 17.624 |
| 38 | 14.000 | .745 | 12.376 | 15.624 |
| 39 | 11.667 | .745 | 10.043 | 13.291 |
| 40 | 10.667 | .745 | 9.043 | 12.291 |

The transition gelling times provide estimates for in vivo applications, which can be adjusted to satisfy the requirements of the delivery method. In this study two methods of delivery were considered, catheter delivery and direct injection. Longer times would be needed for catheter delivery considering the distance travel from the tip to the heart. Hence, the times obtained fall in a reasonable range for catheter delivery. On the other hand, if the delivery method was direct injection the times attained in this experiment might be too high.

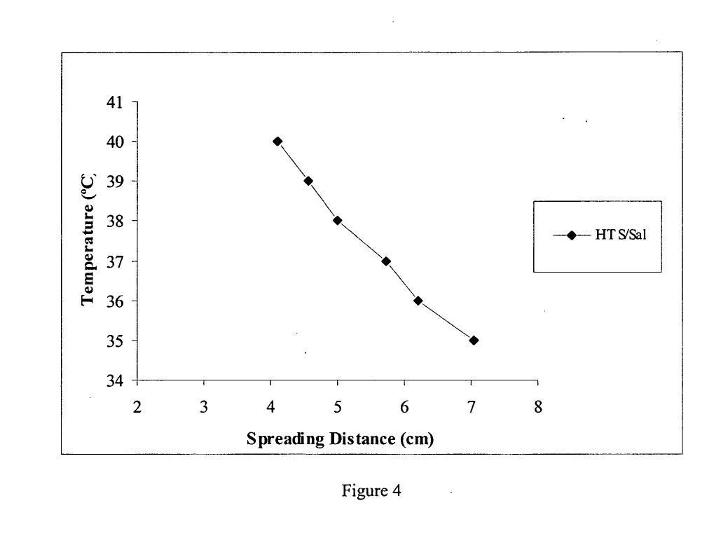

From FIG. 4 it was inferred that spreading distance is inversely proportional to temperature; wherein at higher temperatures the spreading distance decreases. This is due to the increasing vicosity of the gel at higher temperatures (Cabana et al, 1997). The spreading distance means is shown in Table III, where an overall distance range of 4- 7 cm was attained for the specified temperatures. Spreading distances also provide a platform for in vivo applications. When injecting the hydrogel into the infarcted heart certain coverage should be expected in order to have cell proliferation, and differentiation. Furthermore, cells should not be clustered when deliver into the heart since it will hinder mass transport and oxygen flow through the hydrogel matrix. Correlating results from FIG. 1 and FIG. 4, it can be inferred that spreading distances can also be optimized with changes in concentration. Thus, an increase in concentration would render lower spreading distances.

TABLE III
Dependent Variable: Spreading

| | | | 95% Confidence Interval | |
|---|---|---|---|---|
| TEMPERAT | Mean | Std. Error | Lower Bound | Upper Bound |
| 35.00 | 7.033 | .088 | 6.841 | 7.225 |
| 36.00 | 6.167 | .088 | 5.975 | 6.359 |
| 37.00 | 5.667 | .088 | 5.475 | 5.859 |
| 38.00 | 5.033 | .088 | 4.841 | 5.225 |
| 39.00 | 4.533 | .088 | 4.341 | 4.725 |
| 40.00 | 4.133 | .088 | 3.941 | 4.325 |

Cell Viability

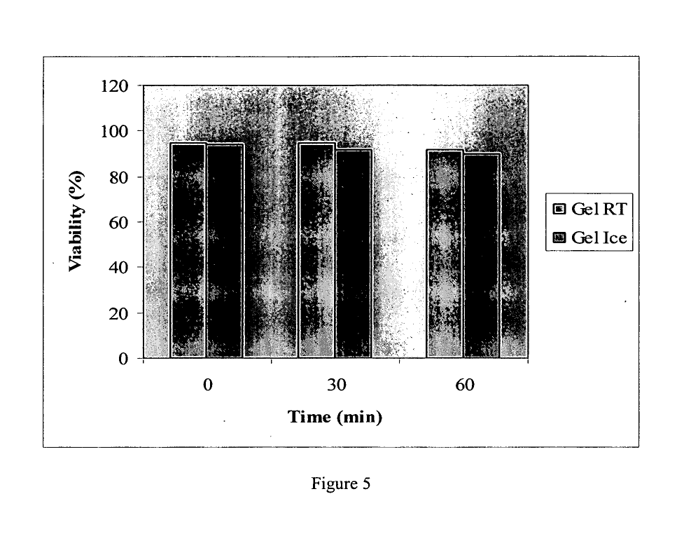

Cell viability in the hydrogel was assessed at three times: 0, 30, and 60 minutes (FIG. 5). The overall assessment rendered high cell viability ratios, at both room temperature and low temperature (2-5° C.). No major differences were noted amongst times and temperatures (FIG. 5). Cell viability ranged from 90% to 95%. The results obtained showed that even for storage periods of one hour at low temperatures viability was not hindered. High viability ratios might be trigger by the presence of HTS in the hydrogel formulation. As mentioned before, HTS is used as an organ preservation solution, thus it is designed to maintain tissue viable for extended periods of time.

pH Dependence

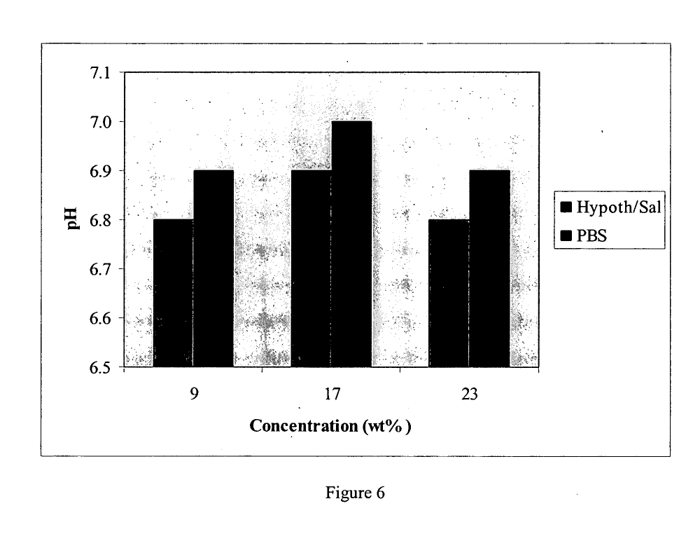

The pH was attained for two hydrogel formulations at three concentrations in order to establish a comparison. As noted in FIG. 1, PBS and HTS/Sal had similar behaviors, hence it was decided to test both solvents. Three concentrations were tested: lowest, highest and selected concentration for hydrogel formulation. Previously, the pH was attained for both solvents: 1) PBS pH=7.1 and 2) HTS/Sal=7.2. The pH results are shown in FIG. 6. After adding the polymer to the solvent a slight decline in pH was observed for PBS dropping from 7.1 to 6.9. A more pronounced decline was observed for HTS/Sal falling from 7.2 to 6.8. The results obtained reflected no significant differences within concentrations for each of the solvents. A trend was noted for both solvents where at low (9 wt %) and high (23 wt %) concentrations the pH was lower, than for the middle concentration (17 wt %). Both formulations presented pH values within neutral limits (pH=7.0). Neutral pH is important when dealing with cell encapsulation considering that most cells stop growing as the pH falls from pH 7.0 to pH 6.5 and start to lose viability between pH 6.5 and pH 6.0.

In vitro Studies

Preliminary in vitro studies of the delivery of the hydrogel matrix through a catheter have been performed. In a water bath, the environment was maintained at a physiological temperature (37±2° C.). Both the saline, which was the control in the study, and the 16% by weight Pluronic™ formulation were maintained at temperature of 4-8° C. In addition, the syringes used for the delivery were also maintained at a chilled temperature of 4-8° C. Cold saline was flushed through the catheter to remove all the air inside the tubing of the catheter. The initial temperature of the saline using one of the wires of the thermocouple or temperature sensor place at the proximal end of the catheter was recorded. The readings were in the range of 13- 19° C. The temperature of the matrix before the delivery was recorded to fall in the range of 5-13° C. Using the second wire of the thermocouple at the distal end of the catheter, the gelation temperature of the saline was recorded to be in the range of 24-26° C. while the temperature of the matrix out from the catheter was in a range 24-29° C. In addition, the travel time for both the saline and the hydrogel matrix through the catheter were recorded where the average gelation time, or the travel time, for the matrix was approximately 17.6 seconds.

The results from this study, using the 16% by weight matrix, provided a range for the intramyocardial injection time to be approximately 13-23 seconds. The hydrogel matrix gelled upon leaving the catheter at room temperature.

TABLE IV
Temperature and Time measurements of the Saline and Hydrogel matrix

| | SALINE | | | HYDROGEL | | |
|---|---|---|---|---|---|---|
| | Temp In | Temp Out | Time | Temp In | Temp Out | Time |
| Trial | (° C.) | (° C.) | (sec) | (° C.) | (° C.) | (sec) |
| 1 | 13.9 | 26.4 | 15 | 13.0 | 24.4 | 17.0 |
| 2 | 15.1 | 24.1 | 8 | 5.6 | 28.5 | 22.0 |
| 3 | 19.1 | 24.4 | 6 | 10.1 | 26.7 | 14.0 |
| Average | 16.0 ± 1 | 25.0 ± 1 | 9.7 ± 1 | 9.6 ± 1 | 26.5 ± 1 | 17.7 ± 1 |

General Information about the catheter (SR200 MyoCath-Bioheart): Volume uptake for the whole length: 0.5 ml; diameter of the catheter: 0.090-0.095 inches; core diameter of catheter: 0.036 inches; length of catheter: 115 cm; time for saline solution to flush: 20 sec; inner shell tubing material: 304-Stainless 20 gauge hypotube w/ braided polyamide; outer shell tubing: Pebax braided with stainless steel.

General method preparations before injection include the following steps. The CardioTak™ (16 wt % Pluronics™; 3 wt % HTS; 0.5 wt % 5000 Da PEG in saline/media) solution is stored in a cold environment (about 4° C.). During the delivery procedure, the solution is kept cold using ice packs in a container. 1.75 mL of cell suspension with media containing 5.0×10⁵ cells/mL is added to the 7 mL CardioTak™ solution. The desired size syringes are placed in the refrigerator to pre-chill before drawing up CardioTak™ solution. The temperature of the environment surrounding the catheter should not exceed 37±2° C. Twenty syringes each containing 0.3 mL CardioTak™ solution containing cells are filled prior to the surgical procedure and maintained at about 4° C. Each CardioTak™ syringe should be free of air bubbles. Due to quick gelation of the polymer, the CardioTak™ syringe is quickly removed from the cold environment, placed on the inject port, and delivered through the catheter to the subject. The catheter is flushed with heparinized saline through the sheath port every 15 minutes. The formulation is injected slowly through the inject port at the rate of about 0.5 mL/min. Too fast of an inject may result in unsuccessful delivery of the desired dose. Recordation of the volume of the CardioTak™ solution going into the catheter is done to ensure accurate delivery of the desired dose of the formulation.

At room temperature, the CardioTak™ solution flows through the catheter with no resistance. When the solution was exposed to 37° C. environment, the following data was recorded.

TABLE V

| | 1^{st} Trial | | 2^{nd} Trial | |
|---|---|---|---|---|
| | Temp (° C.) | Time (sec) | Temp (° C.) | Time (sec) |
| Saline in | 15.9 | 4 | 16.0 | 5 |
| Saline out | 28.8 | | 28.0 | |
| Polymer in | 12.2 | 67 | 13.3 | 51 |
| Polymer out | 25.3 | | 27.9 | |

Cell Delivery Injection Protocols, In Vivo Studies
Materials

New Zealand white rabbits (Harlan), letitium and gold microspheres (BioPal, Inc.) dimethylformamide (DMF), 27 gauge needles, gastight syringes (Hamilton) are all used in the following protocols

Design of the Experimental Procedure

Experimental:

Sixteen healthy New Zealand white rabbits were used. To prepare sample vials, six 1.5 mL eppendorf vials were labeled. For each rabbit, two vials of samples were produced. The first eppendorf vial, called Control, had 300,000 letitium microspheres with 300 μl saline. The second vial, known as Experimental, had of 300,000 gold microspheres and 300 μL of formulation as described above (16 wt % Pluronics®; 3wt % HTS; 0.5wt % 5000 PEG in saline/media).

The in vitro procedure was followed as described above, except DMF was not added for analysis using a spectrophotometer. 27 gauge needles were bent 90°. 250 μL gastight Hamilton syringes, bent needles, and 25 gauge needles were placed in a refrigerator. For collection of samples and tissue, 15 mL conical tubes were prelabeled. Two sets were made; one set was of control tubes and the other set was of experimental tubes. The three vials containing saline, used as a control, were labeled as “Control Dose”, “Needle from Control Dose”, and “Needle from Heart”. The Control Dose means a 50 μL injection of saline with letitium colored microspheres. The vial labeled as “Needles from Control Dose” is the vial containing the microspheres left in the bent needle after the suspension is injected into the Control Dose vial. The third vial labeled as “Needle from Heart” contains the remaining microspheres in the needle cleaned with saline after the suspension is injected in to the left ventricle of the rabbit's heart. Three additional vials, experimental vials containing the polymer, were labeled as “Experimental Dose”, “Needle from Experimental Dose”, and “Needle from Heart”.

Sample Preparation:

160 μL of either a letitium (control) or gold (experimental) microsphere solution (obtained from BioPal, Inc supplied bottle) was drawn into a 250 μL gastight syringe. The supernatant liquid (saline and 0.01% Tween 80) was extracted from each vial/test tube. Next, 400 μL saline was placed in each viautest tube (control or experimental as described above in paragraph [0099]) and each tube was vortexed and centrifuged. The supernatant was removed. This procedure of washing with saline was repeated two more times. The samples were then allowed to dry for 0.75-1 hour. Next, 200 μL saline was added to the control tubes, and 200 μL CardioTak™ was added to the experimental tubes. The prepared samples were stored in a refrigerator until used on the rabbits.

When the rabbit's heart was exposed, a sequence of injections was performed. First, a beaker was filled with saline. Using a 250 μL gastight syringe and a 25 gauge needle, saline was drawn into the syringe and then expelled. This step was performed a minimum of three times to ensure no air bubbles in the needle. For the control studies, a 50 μL dose of saline and the letitium microspheres was drawn into the syringe a minimum of three times to ensure no dilution of the letitium microspheres with residual saline in the syringe. The 25 gauge needle was replaced with a 90° bent 27 gauge needle. One injection as prepared above was injected into a control vial, and a second was injected into the rabbit's left ventricle. The needles used for the two injections were cleaned with saline in the appropriate vials. The polymer (i.e., experimental) injections were performed using the same protocol.

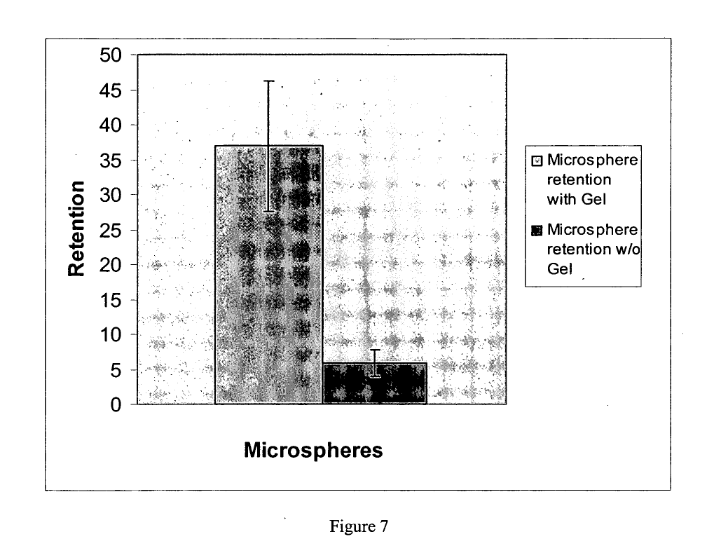

After the injection, the rabbit's heart was removed and divided into three sections—left ventricular free wall, the septum, and the right ventricular free wall. These parts were placed into separate tubes and labeled accordingly. The tissue samples were frozen for a short period of time. Each sample was evaluated for neutron activation processing using procedures known to those of skill in the art. The results of these evaluations are shown in FIG. 7, as an average of 14 of the rabbits. The average cell retention was about 36% with the hydrogel formulation but a mere 5% without, indicating that the hydrogel was effective in delivering and retaining the cells in an in vivo environment.

The foregoing describes and exemplifies the invention but is not intended to limit the invention defined by the claims which follow. All of the arrays and/or methods disclosed and claimed herein can be made and executed without undue experimentation in light of the present disclosure. While the materials and methods of this invention have been described in terms of specific embodiments, it will be apparent to those of skill in the art that variations may be applied to the materials and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those of ordinary skill in the art are deemed to be within the spirit, scope and concept of the invention as defined by the appended claims.

Additional information may be found in the following, all of which are incorporated by reference herein in their entirety.

American Heart Association. Heart Disease and Stroke Statistics—2004 Update. Dallas, Tex.: American Heart Association; 2002. (Online) Available: http://www.americanheart.org/ (Mar. 10, 2003).

Al-Radi et al. (2003) “Cardiac Cell Transplantation: Closer to Bedside.” Ann. Thorac. Surg. 75:S674-7.

Etzion et al. (2001). “Influence of embryonic cardyomyocyte transplantation on the progression of heart failure in a rat model of extensive myocardial infarction.” J. Mol. Cell. Cardiol. 33:1323-30.

Trainini et al. (2003). “Autologous cellular cardiac-implant.” Basic. Appl. Myol. 13(1):39-43.

White et al., “Cardiac cell transplantation”, From Metzger, J. M (ed). Cardiac cell and gene transfer: principles, protocols and applications. Totowa: Humana Press, 2003.

Menasché (2002). “Cell therapy of heart failure.” C.R. Biologies 325:731-738.

Taylor (2001) “Cellular cardiomyoplasty with autologous skeletal myoblasts for ischemic heart disease and heart failure.” Curr. Control Trials Cardiovasc. Med. 2:208-10.

Marler “Skeletal muscle”, From Atala A, Lanza R. P., (ed.). Methods of tissue engineering, Second Edition. San Diego: Academic Press, 2002.

Sagen et al. “Transplantation of encapsulated cells into the central nervous system”, From Kühtreiber W. M., Lanza R, Chick W, (ed.). Cell Encapsulation Technology and Therapeutics. Boston: Birkhäuser, 1999.

Alexandridis et al. (1995). “(Poly(ethylene oxide)-poly(propylene oxide)-poly(ethylene oxide) triblock copolymer surfactants in aqueous solutions and at interfaces. Thermodynamics, structure, dynamics, and modeling.” Coll. Surf. A 96:1-46.

Bohorquez et al. (1999). “A study of the temperature dependent micellization of Pluronic F 127.” J. Coll. Inter. Science 216:34-40.

Matthew et al. (2002). “Effect of mammalian cell culture medium on the gelation properties of Pluronic™ F127.” Biomaterials 23:4615-19.

Pluronic and Tetronic Surfactants, Technical Brochure, BASF Corp., Parsippany, N.J., 1999.

Hoffman (2002). “Hydrogels for biomedical applications.” Adv. Drug Del. Rev. 43:3-12.

Kabanov et al. (2002). “Pluronic™ block copolymers as novel polymer therapeutics for drug and gene delivery.” J. Cont. Rel. 82:189-212.

http://ocw.mit.edu/ocw/web/index.htm Molecular Principles of Biomaterials, Spring 2003, Lecture 8: Physical Hydrogels.

Moore et al. (2000). “Experimental investigation and mathematical modeling of Pluronic® F127 gel dissolution: drug release in stirred systems”. J. Cont. Rel. 67:191-202.

Schmolka (1972). “Artificial skin I. Preparation and properties of pluronics F-127 for treatment of burns.” J. Biomed. Mat. Res. 6:571-582.

Skehan. “Cytotoxicity and cell growth analysis”, From Celis J (ed.). Cell Biology: A Laboratory Handbook. San Diego: Academic Press, 1998.

Freed et al. “Culture environments: cell-polymer-bioreactor systems”, From Atala, A, Lanza, R P, (eds.). Methods of tissue engineering. San Diego: Academic Press, 2000.

Gao et al. (1998). “Surface hydrolysis of poly (glycolic acid) meshes increases the seeding density of vascular smooth muscle cells.” J. Biomed. Mater. Res. 42:417-24.

Radisic et. al. (2003). “High density seeding of myocite cells for cardiac tissue engineering.” Biotechnol. Bioeng. 82:403-414.

Mooney et al. (1998). Optimizing seeding and culture methods to engineer smooth muscle tissue on biodegradable polymer matrices.” Biotechnol Bioeng; 57:46-54.

Martini. Fundamentals of Anatomy and Physiology. New Jersey: Prentice Hall 2001.

Lum et al. “Injectable hydrogels for cartilage tissue engineering”, From Ashammakhi N and Ferretti P (eds). Topics in tissue engineering, 2003.

Chiu. “Cardiac cell transplantation: the autologous skeletal myoblast implantation for myocardial regeneration”, From Karp R. B (ed.). Advances in cardiac surgery, volume 11. Mosby Inc., 1999.

Reinecke et al., “Cell grafting for cardiac repair”, From Metzger, J. M (ed). Cardiac cell and gene transfer: principles, protocols and applications. Totowa: Humana Press, 2003.

Gulbins et al. (2002). “Cell transplantation—a potential therapy for cardiac repair in the future?” The heart surgery forum 5(4): editorial.

Taylor et al. (1997). “Delivery of primary autologous skeletal myoblasts into rabbit heart by coronary infusion: a potential approach to myocardial repair.” Proc. Amer. Assoc. Amer. Phys. 109(3): 245-253

Langer et al. (1993). “Tissue engineering.” Science 260:920-926.

Chaignaud et al. “The history of tissue engineering using synthetic biodegradable polymer scaffolds and cells.” From Atala A., Mooney D., Vacanti J. P., and Langer R (eds.). Synthetic Biodegradable Polymer Scaffolds, Boston: Birkhäuser; 1997.

King et al. “Cell immobilization technology: and overview.” From Goosen F. A. (ed.). Fundamentals of animal cell encapsulation and immobilization. Boca Raton, Fla.: CRC Press; 1993.

Okhamafe et al. “Modulation of membrane permeability” From Kuthreiber W. M. et al. (ed.). Cell encapsulation technology and therapeutics, Boston: Birkhäuser; 1999.

Christenson et al. “Biomedical applications of immobilized cells.” From Goosen F. A. (ed.). Fundamentals of animal cell encapsulation and immobilization. Boca Raton, Fla.: CRC Press; 1993.

Park et al. “Hydrogels in bioaplications” From Park K., Shalaby W. S., and Park H. (eds.). Biodegradable hydrogels for drug delivery. Lancaster, Pa.: Technomic Publishing C., Inc 1993.

Elisseeff et al. (1999). “Transdermal photopolymerization of poly(ethylene oxide)-based injectable hydrogels for tissue engineered cartilage.” Plast. Reconstr. Sur. 104(4):1014-1022.

Cao et al. (1998). “Comparative study of the use of poly (glycolic acid), calcium alginate and pluronics in the engineering of autologous porcine cartilage.” J. Biomater. Sci. Polym. 9:474-87.

Rosiak et al. (1999). “Hydrogels and their medical applications.” Nucl. Instr. Meth. Phys. Res. B. 151: 56-64.

Yaffe et al. (1977). “Serial passaging and differentiation of myogenic cells isolated from dystrophic mouse muscle.” Nature. 270 (5639):725-727

Jeon et al. (2002) “Microviscosity in poly (ethylene oxide)-polypropylene oxide-poly(ethylene oxide) block copolymers probed by fluorescence depolarization kinetics.” J. Poly. Sci. Part B: Pol. Phys. 40: 2883-2888.

Banasiak et al. (1999). “Comparison between clogenic, MTT, and SRB assays for detemining radiosensitivity in a panel of human bladder cancer cell lines and a uretral cell line.” Rad. Onc. Inv. 7:77-85

Martin et al. (2004). “The role of bioreactors in tissue engineering.” Trends in Biotech. 22(2):80-87.

“Hydrogels” From Ottenbrite R. M., Huang S. J., and Park K. (eds.). Hydrogels and biodegradable polymers for bioaplications. Washington D.C. American chemical society; 1996.

Cohn et al. (2003). “Improved reverse thermo responsive polymeric systems.” Biomaterials 24: 3707-3714.

Marler et al. (1998). “Transplantation of cells is matrices for tissue regeneration.” Ad. Drug. Del. Rev. 33: 165-182.

Smith (2002). “Will tissue engineering become the 21st century's answer to cardiovascular disease?” Heart, Lung and Cir. 11: 135-137

Pandit et al. (2000). “Effect of salts on the micellization, clouding, and solubilization behavior of Pluronic F127 solutions.” J. Coll. Int. Sci. 222:213-220

Baust et al. (2002). “Modulation of the cryopreservation cap: elevated survival with reduced dimethyl sulfoxide concentration.” Cryobiology. 45:97-108.

Cabana et al. (1997). “Study of the gelation process of polyethylene oxidea-polypropylene oxideb-polyethylene oxidea copolymer (poloxamer 407) aqueous solutions.” Int. Sci. 190:307-312

What is claimed is:

1. A formulation for controlled cell delivery comprising a prepolymer, a temperature protectant, and an additive, wherein the prepolymer is about 12 to about 18 wt % of the total formulation and the additive is about 0.2 to about 1 wt % of the total formulation, wherein the prepolymer and additive are capable of reacting at a temperature of about 33 to about 40° C. to form a hydrogel.

2. The formulation of claim 1, further comprising a cell suspension.

3. The formulation of claim 2, wherein the cell is selected from the group consisting of smooth muscle cells, endothelial cells, epithelial cells, fibroblasts, skeletal myoblasts, bile duct cells, pancreatic islet cells, thyroid cells, parathyroid cells, adrenal cells, hypothalamic cells, pituitary cells, ovarian cells, testicular cells, salivary cells, chondrocytes, hepatocytes, enterocytes, nerve cells, cardiac cells, and kidney cells.

4. The formulation of claim 2, wherein the cells are skeletal myoblast cells.

5. The formulation of claim 1, wherein the prepolymer is a triblock copolymer polyethylene oxide-polypropylene oxide-polyethylene oxide (PEO-PPO-PEO).

6. The formulation of claim 5, wherein the PEO-PPO-PEO has a molecular weight of about 10,000 to about 14,000 Da and the ratio of PEO to PPO is about 1.3:1 to about 2:1.

7. The formulation of claim 1, wherein the additive is polyethylene glycol.

8. The formulation of claim 1, wherein the additive is about 0.2 to about 0.5 wt %.

9. The formulation of claim 1, wherein the temperature protectant is HypoThermosol®.

10. A hydrogel prepared from the formulation of claim 2.

11. The hydrogel of claim 10, wherein the cells have a cell retention rate of at least 20%.

12. The hydrogel of claim 10, wherein the prepolymer is a triblock copolymer polyethylene oxide-polypropylene oxide-polyethylene oxide (PEO-PPO-PEO), the additive is polyethylene glycol, and the temperature protectant is HypoThermosol®, and wherein the PEO-PPO-PEO has a molecular weight of about 12,000 to about 14,000, the polyethylene glycol is about 0.5 to about 0.7 wt % of the formulation, and the cell suspension comprises skeletal myoblast cells.

13. The hydrogel of claim 12, wherein the hydrogel is biodegradable.

14. A method of delivering cells to a subject in need thereof comprising contacting a formulation to said subject, wherein the formulation comprises a prepolymer, a temperature protectant, an additive, and a cell suspension, wherein the prepolymer is about 12 to about 16 wt % and the additive is about 0.2 to about 1 wt %, wherein the prepolymer and additive react at a temperature of about 30 to about 40° C. to form a hydrogel.

15. The method of claim 14, wherein the prepolymer is a triblock copolymer polyethylene oxide-polypropylene oxide-polyethylene oxide (PEO-PPO-PEO).

16. The method of claim 15, wherein the PEO-PPO-PEO has a molecular weight of about 12,000 to about 13,500 Da and a ratio of PEO to PPO of about 2:1.

17. The method of claim 14, wherein the cell suspension is a suspension of myoblast cells.

18. The method of claim 14, wherein the subject is a human or animal subject.

19. The method of claim 18, wherein the subject suffers from a cardiovascular disease.

20. The method of claim 19, wherein the cardiovascular disease is myocardial infarction.
